(19) United States
(12) Patent Application Publication
(10) Pub. No.: US 2024/0335303 A1
(43) Pub. Date: Oct. 10, 2024

(54) UNIBODY CYLINDER CONSTRUCTION FOR MICROPROCESSOR-CONTROLLED PROSTHETIC KNEE

(71) Applicant: College Park Industries, Inc., Warren, MI (US)

(72) Inventors: Jacob Michael Drews, Washington, MI (US); Aaron John Taszreak, China, MI (US); Anna Mae Marlatt, Waterford, MI (US)

(21) Appl. No.: 18/612,328

(22) Filed: Mar. 21, 2024

Related U.S. Application Data
(60) Provisional application No. 63/495,022, filed on Apr. 7, 2023.

(51) Int. Cl. A61F 2/74; A61F 2/64; A61F 2/70

(52) U.S. Cl. CPC A61F 2/741; A61F 2/64; A61F 2/748; A61F 2002/704

(57) ABSTRACT

A unibody (e.g., seamless single-piece, monolithic) cylinder for a prosthetic knee defines a hydraulic cylinder in which a piston travels, defines fluid pathways for a hydraulic fluid, and defines bores that house embedded gearmotors and valves operable to actively control a damping rate of flexion and extension of the prosthetic knee. The unibody cylinder also houses sensors and electronics (e.g., circuit board) and cabling connecting them to the gearmotors. The unibody cylinder minimizing surfaces or junctions that come into contact with the hydraulic fluid to reduce sealing requirements, inhibit (e.g., prevent) hydraulic leaks and facilitate waterproofing of the unibody cylinder.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims priority benefit of U.S. Provisional Application No. 63/495,022, filed Apr. 7, 2023, the entirety of which is hereby incorporated by reference herein.

### BACKGROUND

### Field

The present disclosure relates generally to a unibody actuation mechanism for a prosthetic joint, more specifically, a prosthetic knee.

### Description of the Related Art

Prosthetics are used to replace and restore the functionality of amputated natural body parts. Microprocessor-controlled prosthetic knees may use gearmotors for controlling the rotation of hydraulic valves. In existing products, the motors, gearboxes, and valves are often housed in a separate unit attached to the hydraulic cylinder. Designs requiring a separate unit for housing the valves and gear motors disadvantageously create more surfaces and openings through which hydraulic oil passes, increasing the likelihood for hydraulic oil leaks. To accommodate the increased risk in leaks, numerous seals are required to prevent hydraulic oil leaking out of the system. An additional disadvantage of this type of design is that the additional unit adds weight to the prosthetic knee. Further, in some devices, the additional housing component may increase the width of the prosthetic knee to be wider overall but doesn't add additional length, complicating the hydraulic flow path and making it difficult to bleed the system of air.

In devices where the motor is embedded into a hydraulic cylinder, hydraulic fluid is directed through one of two manually adjusted pathways by use of a switch. This does not allow for real-time adjustments to the resistances such that the prosthesis cannot actively adapt to the user's gait in real-time.

### SUMMARY

The embodiments disclosed herein each have several aspects, no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices, and methods for a unibody cylinder construction with an embedded valve-gearmotor system for a microprocessor-controlled prosthetic knee.

The following disclosure describes non-limiting examples of some embodiments. Other embodiments of the disclosed systems and methods may or may not include the features described herein. Moreover, disclosed advantages and benefits can apply only to certain embodiments of the invention and should not be used to limit the disclosure.

In some aspects, the techniques described herein relate to a prosthetic knee, including: a unibody hydraulic cylinder extending between a proximal end and a distal end, including: a central bore configured to movably receive a piston attached to a piston rod; one or more bores configured to house a motorized valve system including a valve; one or more pathways via which hydraulic fluid flows, and one or more bores configured to house cabling connecting the motorized valve system to a circuit board, wherein the central bore and one or more pathways are in fluid communication with each other via the valve.

In some aspects, the techniques described herein relate to a prosthetic knee, wherein the one or more bores configured to house a motorized valve system are two bores, each configured to house a motorized valve system with a valve.

In some aspects, the techniques described herein relate to a prosthetic knee, wherein the unibody hydraulic cylinder has a single sealing surface at one end thereof.

In some aspects, the techniques described herein relate to a prosthetic knee, wherein the single sealing surface is at the distal end of the unibody hydraulic cylinder.

In some aspects, the techniques described herein relate to a prosthetic knee, wherein the central bore, one or more bores configured to house the motorized valve system, one or more pathways and one or more bores configured to house cabling extend at least partially along parallel axes along a length of the unibody hydraulic cylinder.

In some aspects, the techniques described herein relate to a prosthetic knee, wherein the central bore has a larger diameter than the one or more bores configured to house the motorized valve system.

In some aspects, the techniques described herein relate to a prosthetic knee, wherein the one or more bores configured to house the motorized valve system has a larger diameter than the one or more pathways.

In some aspects, the techniques described herein relate to a prosthetic knee, wherein the one or more pathways are two pathways.

In some aspects, the techniques described herein relate to a unibody hydraulic cylinder for a prosthetic knee, including: a central bore configured to movably receive a piston attached to a piston rod; one or more bores configured to house a motorized valve system including a valve; one or more pathways via which hydraulic fluid flows, and one or more bores configured to house cabling connecting the motorized valve system to a circuit board, wherein the central bore and one or more pathways are in fluid communication with each other via the valve.

In some aspects, the techniques described herein relate to an unibody hydraulic cylinder, wherein the one or more bores configured to house a motorized valve system are two bores, each configured to house a motorized valve system with a valve.

In some aspects, the techniques described herein relate to an unibody hydraulic cylinder, further including a single sealing surface at one end of the unibody hydraulic cylinder.

In some aspects, the techniques described herein relate to an unibody hydraulic cylinder, wherein the single sealing surface is at a distal end of the unibody hydraulic cylinder.

In some aspects, the techniques described herein relate to an unibody hydraulic cylinder, wherein the central bore, one or more bores configured to house the motorized valve system, one or more pathways and one or more bores configured to house cabling extend at least partially along parallel axes.

In some aspects, the techniques described herein relate to an unibody hydraulic cylinder, wherein the central bore has a larger diameter than the one or more bores configured to house the motorized valve system.

In some aspects, the techniques described herein relate to an unibody hydraulic cylinder, wherein the one or more bores configured to house the motorized valve system has a larger diameter than the one or more pathways.

In some aspects, the techniques described herein relate to an unibody hydraulic cylinder, wherein the one or more pathways are two pathways.

In some aspects, the techniques described herein relate to a device for actuation of a prosthetic, including: an elongate hollow body defining one or more bores, a hydraulic system, the hydraulic system including: a piston; hydraulic fluid; an accumulator; and one or more check valves, and a motorized valve system, the motorized valve system including: one or more motors, and one or more valve cartridges, the one or more valve cartridges being operably connected to the one or more motors, wherein the hydraulic system and motorized valve system are housed within the one or more bores, at the elongate hollow body having a single sealing surface.

In some aspects, the techniques described herein relate to a device, wherein the prosthetic is a prosthetic knee or a prosthetic foot.

In some aspects, the techniques described herein relate to a device, further including one or more Hall effect sensors.

In some aspects, the techniques described herein relate to a device, where the one or more Hall effect sensors transmit one or more signals relating to position of the valve cartridges to the one or more motors.

In some aspects, the techniques described herein relate to a device, wherein the accumulator is a spring-based accumulator.

In some aspects, the techniques described herein relate to a device, wherein the one or more bores includes a first bore housing the hydraulic system, and at least one additional bore separate from the first bore and configured to house the motorized valve system.

In some aspects, the techniques described herein relate to a device, further including: a piston rod, and a connector portion, the connector portion coupled to a proximal end of the piston rod and configured to rotatably couple to a frame of the prosthetic.

In some aspects, the techniques described herein relate to a device, wherein the hydraulic fluid is included of one or more of glycol ether, organophosphate ester, polyalphaolefin, a propylene glycol, a silicone oil, or NaK-7.

In accordance with one aspect of the disclosure, a unibody (e.g., single piece, monolithic) cylinder is provided for a hydraulic prosthetic knee (e.g., a processor controlled hydraulic prosthetic knee). The unibody cylinder includes (e.g., defines) a hydraulic cylinder in which a piston travels (e.g., to move the knee between flexion and extension positions), cylinders or passages that house valves and gearmotors, and passages through which the hydraulic fluid flows. The unibody cylinder provides a self-contained unit that houses the hydraulics (e.g., hydraulic fluid, valves, gearmotors, piston, piston rod), advantageously reducing the weight of the hydraulic prosthetic knee and reducing the risk of hydraulic leaks by reducing (e.g., minimizing) the number of surfaces or junctions through which hydraulic fluid passes.

In one aspect of the present disclosure, a prosthetic knee is described. The prosthetic knee may include a unibody hydraulic cylinder extending between a proximal end and a distal end. The hydraulic cylinder may comprise a central bore configured to movably receive a piston attached to a piston rod, one or more bores configured to house a motorized valve system including a valve, one or more pathways via which hydraulic fluid flows, and one or more bores configured to house cabling connecting the motorized valve system to a circuit board. The central bore and one or more pathways may be in fluid communication with each other via the valve. In some embodiments, the one or more bores configured to house a motorized valve system are two bores, each configured to house a motorized valve system with a valve. In some embodiments, the unibody hydraulic cylinder has a single sealing surface at one end. In some embodiments, the single sealing surface is at the distal end of the unibody hydraulic cylinder. In some embodiments, the central bore, one or more bores configured to house the motorized valve system, one or more pathways and one or more bores configured to house cabling extend at least partially along parallel axes along a length of the unibody hydraulic cylinder. In some embodiments, the central bore has a larger diameter than the one or more bores configured to house the motorized valve system. In some embodiments, the one or more bores configured to house the motorized valve system has a larger diameter than the one or more pathways. In some embodiments, the one or more pathways are two pathways.

In another aspect of the present disclosure, a unibody hydraulic cylinder for a prosthetic knee is described. The unibody hydraulic cylinder may include a central bore configured to movably receive a piston attached to a piston rod, one or more bores configured to house a motorized valve system including a valve, one or more pathways via which hydraulic fluid flows, and one or more bores configured to house cabling connecting the motorized valve system to a circuit board. The central bore and one or more pathways may be in fluid communication with each other via the valve. In some embodiments, the one or more bores configured to house a motorized valve system are two bores, each configured to house a motorized valve system with a valve. In some embodiments, the unibody hydraulic cylinder may include a single sealing surface at one end of the unibody hydraulic cylinder. In some embodiments, the single sealing surface is at a distal end of the unibody hydraulic cylinder. In some embodiments, the central bore, one or more bores configured to house the motorized valve system, one or more pathways and one or more bores configured to house cabling extend at least partially along parallel axes. In some embodiments, the central bore has a larger diameter than the one or more bores configured to house the motorized valve system. In some embodiments, the one or more bores configured to house the motorized valve system has a larger diameter than the one or more pathways. In some embodiments, the one or more pathways are two pathways.

In another aspect of the present disclosure, a device for actuation of a prosthetic is described. The device may include an elongate hollow body defining one or more bores, and a hydraulic system including a piston, hydraulic fluid, an accumulator, and one or more check valves. The device may further include a motorized valve system including one or more motors, and one or more valve cartridges operably connected to the one or more motors, wherein the hydraulic system and motorized valve system are housed within the one or more bores, at the elongate hollow body having a single sealing surface. In some embodiments, the device may be used for actuation of a prosthetic knee or a prosthetic foot. In some embodiments, the device may include one or more Hall effect sensors. In some embodiments, the one or more Hall effect sensors transmits one or more signals relating to position to the one or more motors. In some embodiments, the accumulator is a spring-based accumulator. In some embodiments, the one or more bores includes a first bore housing the hydraulic system, and at least one additional bore separate from the first bore and configured to house the motorized-valve system. In some embodiments, the device may include a piston rod, and a connector portion coupled to a proximal end of the piston rod and configured to rotatably couple to a prosthetic joint. In some embodiments, the hydraulic fluid is comprised of one or more of glycol ether, organophosphate ester, polyalphaolefin, a propylene glycol, a silicone oil, or NaK-7.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

The following drawings are for illustrative purposes only and show non-limiting embodiments. Features from different figures may be combined in several embodiments.

FIG. 1 is a side view of an example of a prosthetic knee containing a unibody cylinder.

FIG. 2 is a front view of an example unibody cylinder with an embedded valve-gearmotor system.

FIG. 3 is a front view of an example of the unibody cylinder with the cylinder wall removed to show the valve-gear motor system.

FIG. 4 is a rear view of an example of unibody cylinder with the cylinder wall removed to show the hydraulic system.

FIG. 5A-5C are various front cross-sectional views of an example of the cylinder body.

FIG. 6 is a bottom view of an example of the cylinder body.

FIG. 7 is partially exploded bottom view of an example of the cylinder showing the single sealing surface.

FIG. 8 is a side cross-section view of an example of the cylinder body showing the single electrical connection.

### DETAILED DESCRIPTION

The following detailed description is directed to certain specific embodiments of prosthetic devices and methods. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. The embodiments of the invention, examples of which are illustrated in the accompanying drawings, are set forth in detail below. Wherever possible, the same reference numbers are used throughout the drawings to refer to the same or like parts.

Microprocessor-controlled hydraulic prosthetic knees are an ideal solution for controlling the rotation of the prosthetic knee joint for above knee amputees. These systems employ one or more microprocessors and various sensors that detect, respond, and react to the bending of the knee joint by modifying the hydraulic resistance in both flexion and extension directions.

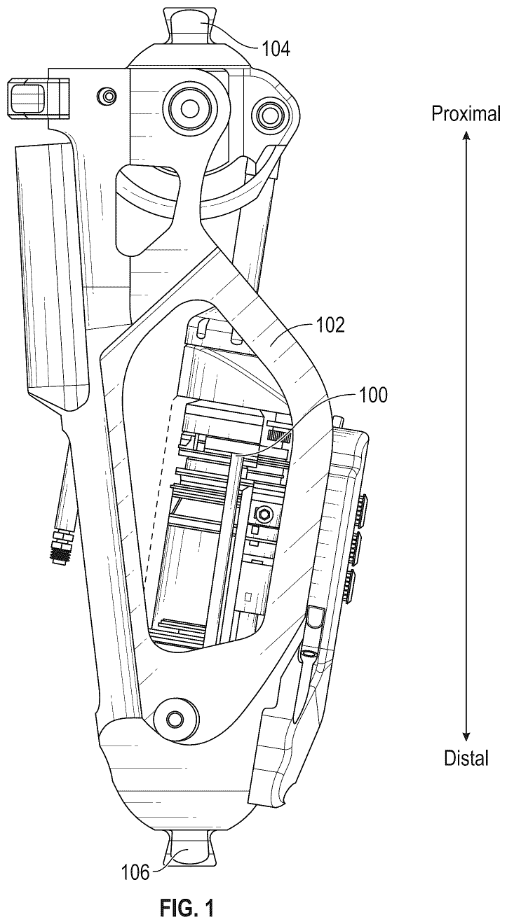

FIG. 1 is a side view of an example of a prosthetic knee 102 containing an embodiment of the unibody cylinder construction 100 described herein. The proximal end of the prosthetic knee 102 may include a proximal connector 104 (e.g., pyramid connector) for connecting the prosthetic knee to an upper leg prosthetic device (e.g., a socket worn by an amputee over their upper leg). The distal end of the prosthetic knee 102 may include a distal connector 106 (e.g., pyramid connector) for connecting the prosthetic knee to a lower leg prosthetic device (e.g., a pylon that couples to a prosthetic foot, a prosthetic foot). The unibody cylinder construction 100 includes a unibody cylinder body 205 (e.g., that is monolithic, a single seamless piece or part).

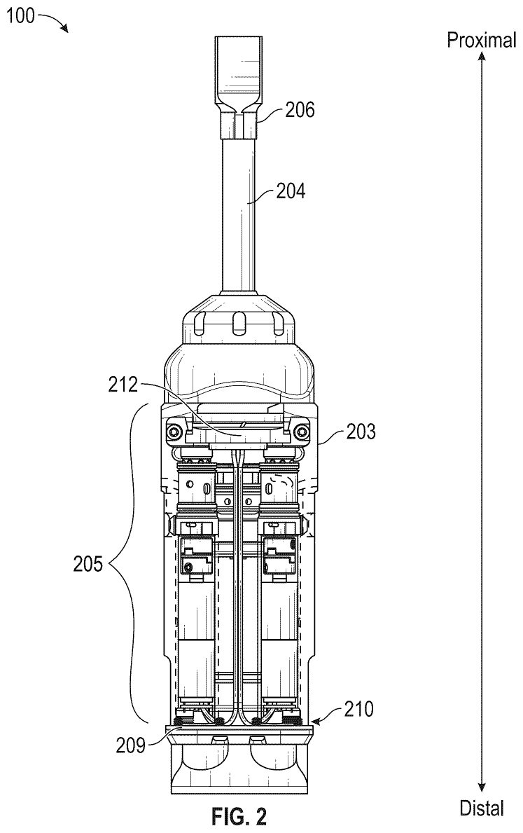

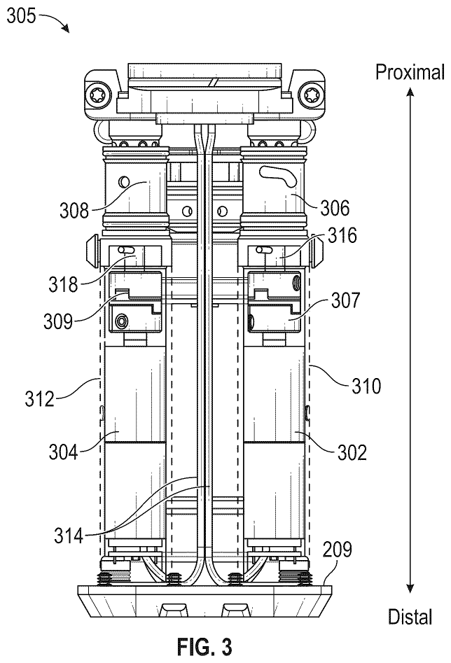

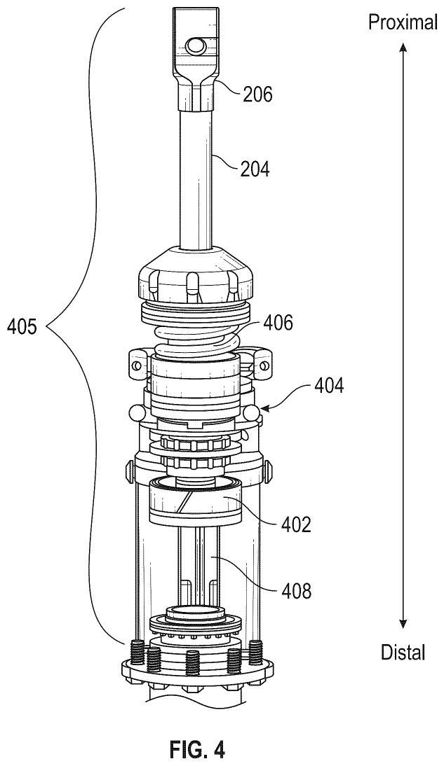

FIG. 2 is a front isolated view of the unibody cylinder construction 100. A proximal connector 206, which allows the unibody cylinder construction 100 to be rotatably coupled to the prosthetic knee 102 is attached to a piston rod 204. The unibody cylinder construction 100 may include a valve-gear motor system 305 and a hydraulic system 405 (see FIGS. 3-4). The hydraulic system 405 may include, for example, one or more of a piston 402 attached to the piston rod 204, check valves 504, hydraulic fluid (e.g., glycol ether, organophosphate ester, polyalphaolefin, a propylene glycol, a silicone oil, NaK-7, etc.) and an accumulator 404, among other components as described in more detail with respect to FIG. 4. The cylinder body 205 may house, for example, one or more valve cartridges 306 and 308, one or more motor couplers 307 and 309, and one or more gearbox and motor assemblies (i.e., gearmotors 302 and 304), among other components as described in more detail with respect to FIG. 3. The proximal end of piston rod 204 may extend from the proximal end of cylinder body 205, while the distal end of the piston rod 204, the piston 402, check valves 504, accumulator 404 and valve-gear motor system 305 may be housed in the cylinder body 205, surrounded by the cylinder wall 203. Between the cylinder base 210 at the distal end of the cylinder body 205, there is a single sealing surface 209 at the interface of the cylinder base 210 and the cylinder body 205, advantageously minimizing the likelihood of hydraulic fluid leakage. The cylinder body 205 may be made of or include any suitable material such as, for example, aluminum, stainless steel, titanium, and the like. A sensor board 212 (e.g., printed circuit board or PCB) housing one or more sensors may be secured to the front of the cylinder body 205. The one or more sensors may include, for example, position sensor(s) (e.g., Hall effect sensor(s)), a magnetometer or magnetic sensor, an accelerometer, a gyroscope and/or other sensors. Feedback signals from the sensors maybe transmitted to the gearmotors 302 and gearmotors 304 via electrical leads (see 314 in FIG. 3).

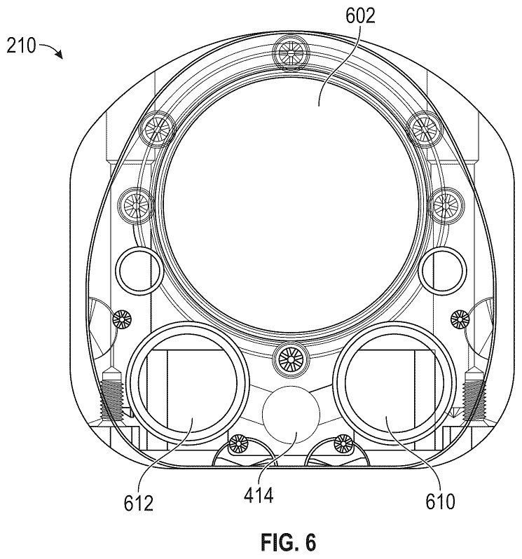

FIG. 3 is a front view of the cylinder body 205 where the cylinder wall 203 is removed to show the valve-gear motor system 305. Motor sleeves 310 and 312 extend proximally from the single sealing surface 209 at the base of the valve-gear motor system 305 and house the motor couplers 307, 309 and gearmotors 302, 304. The motor sleeves 310 and 312 electrically isolate gearmotors 302 and 304 as well as prevent rotation of gearmotors 302 and 304. Additionally, motor sleeves 310 and 312 advantageously reduce vibration and noise from the gearmotors 302 and 304 inside of the cylinder body 205. The motor sleeves 310 and 312 are inserted within the hollow shafts or bores 610, 612 which are defined by (e.g., formed in) the unibody cylinder body 205 as described with respect to FIG. 6. Gearmotors 302 and 304 are activated in response to feedback from the various sensors and accordingly rotate the valve cores 316 and 318 of valve cartridges 306 and 308, respectively. Gearmotor 302 is located at a distal end of motor sleeve 310 and motor coupler 307 is located at a proximal end of motor sleeve 310 and is activated during flexion of the prosthetic knee. Gearmotor 304 is located at a distal end of motor sleeve 312 and motor coupler 309 is located at a proximal end of motor sleeve 312 and is activated during extension of the prosthetic knee. Motor couplers 307 and 309 connect gearmotors 302 and 304 and valve cartridges 306 and 308, respectively. As the valve cores 316 and 318 are rotated by the gearmotors 302, 304 via the motor couplers 307, 309 based on information from the various sensors of the sensor board 212 and other sensors of the prosthetic knee 102 that provide information indicative of the user's gait, the size of a valve orifice in the valve cartridges 306, 308 via which the hydraulic fluid passes can be varied, which in turn changes the hydraulic resistance (e.g., provided by the valve cartridges 306, 308). The valves (e.g., valve orifice size) of the valve cartridges 306, 308 can be controlled independently of each other (e.g., via their respective gearmotors 302, 304), thereby providing independent control of hydraulic resistance in extension and in flexion for the prosthetic joint (e.g., the prosthetic knee). In some embodiments, the valve cartridges 306 and 308 may be insulated (e.g., with one or more layers of insulation material that surrounds the valve cartridges 306 308, motor couplers 307, 309 and/or gearmotors 302, 304) to advantageously inhibit (e.g., prevent) rotation within the hollow shafts or bores 610 and 612, vibration, and/or noise. The insulation may also provide electrical isolation of the valve cartridges 306 308, motor couplers 307, 309 and/or gearmotors 302, 304 from the cylinder body.

FIG. 4 is a rear view of the unibody cylinder construction 100 with the cylinder wall 203 removed to show the hydraulic system 405. In some embodiments, the accumulator 404 of the hydraulic system 405 is a spring-based accumulator, such that when the energy accumulated in the accumulator 404 is released, force is applied on the piston 402 causing the piston 402 to move to the proximal end of the chamber 408 to facilitate (e.g., aid in) extension of the knee. In other embodiments, other accumulator types may be used. When the sensors detect the prosthetic knee is flexing during gait or other activities, feedback signals are sent to gearmotor 302 which can control the valve orifice size in the valve cartridge 306 to control the hydraulic resistance provided by the valve cartridge 306, allowing hydraulic fluid to drain from the hydraulic chamber 408 as the piston 402 moves in the distal direction, compressing a spring 406 coupled to the accumulator 404. Such motion in turn causes the piston rod 204 to move distally into the cylinder body 205 to effect flexion of the prosthetic knee. The hydraulic chamber 408 has less volume for the hydraulic fluid proximal of the piston 402 (e.g., when the knee is in flexion) due to the piston rod 204 taking up space in the hydraulic chamber 408 as compared to the volume available to the hydraulic fluid distal of the piston 402 (e.g., when the knee is in extension). Therefore, to account for this imbalance, as the piston 402 moves distally within the cylinder body 205 during flexion of the prosthetic knee, the hydraulic fluid flows into a volume of the hydraulic chamber 408 proximal of the piston 402 and charges the accumulator 404 (e.g., applies a force on the spring-loaded accumulator).

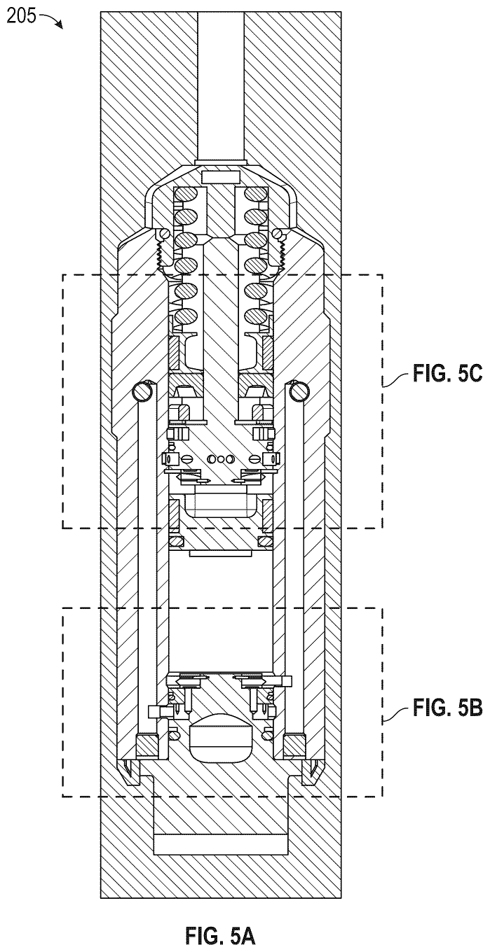

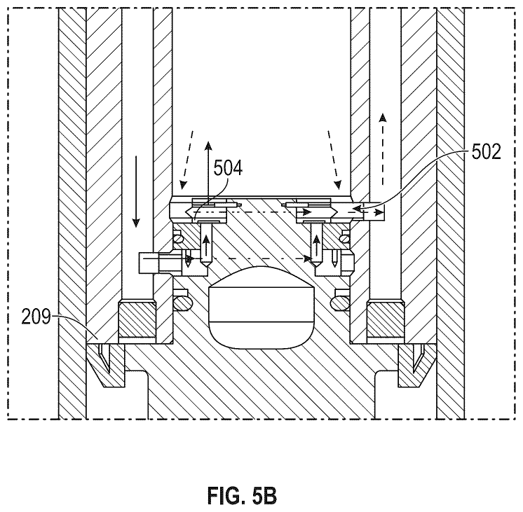

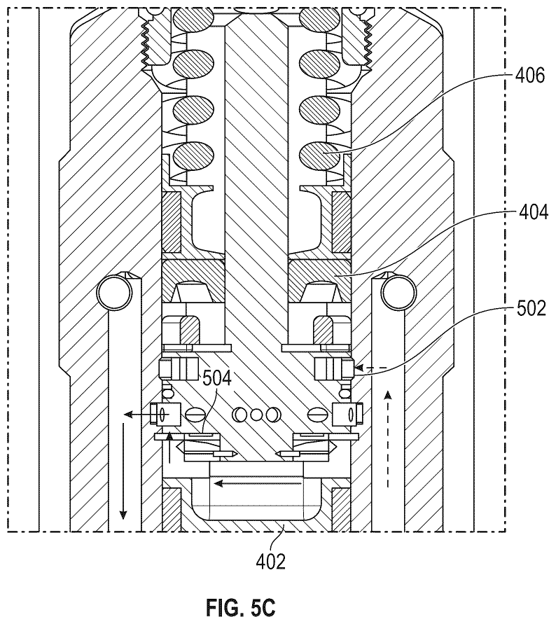

FIGS. 5A-5C show various cross-sections of the cylinder body 205. FIG. 5A is a front cross-sectional view of the cylinder body 205 (e.g., seamless single-piece, monolithic cylinder body). FIG. 5B is an up-close view of region “B” in FIG. 5A and FIG. 5C is an up-close view of region “C” in FIG. 5A. As a user extends the knee, the load applied to the piston 402 (e.g., by the user via the knee joint) causes the piston 402 to travel in the proximal direction within the cylinder body 205. Hydraulic fluid flows through check valves 504 and fills the hydraulic chamber 408 as indicated by the red arrows, for example on the left side of FIGS. 5B and 5C. For simplicity, flow through the check valves 504 are shown on the left sides of the cross-sections in FIG. 5B and FIG. 5C, but one of skill in the art will recognize that the flow through the check valve 504 would also occur on the right side of the cross-section in FIG. 5B and FIG. 5C as shown (e.g., because the check valve is a spring-loaded annular plate). The dashed red line in FIG. 5B indicates flow that goes around a hub portion at the bottom of the cylinder body 205 from left to right.

As a user flexes the knee, the load applied to the piston 402 causes the piston 402 to travel within the cylinder body 205 in the distal direction. Hydraulic fluid is forced out of the hydraulic chamber 408 through passages 502 in the direction of black arrows indicated on the right hand side of FIGS. 5B and 5C. For simplicity, flow through the passage 502 is shown on the right side of the cross-sections in FIGS. 5B and 5C, but one of skill in the art will recognize that the flow through the passage 502 would also occur on the left side of the cross-section in FIG. 5B as shown by black arrows. The dashed black line in FIG. 5B indicates flow that goes around a hub portion at the bottom of the cylinder body 205 from left to right.

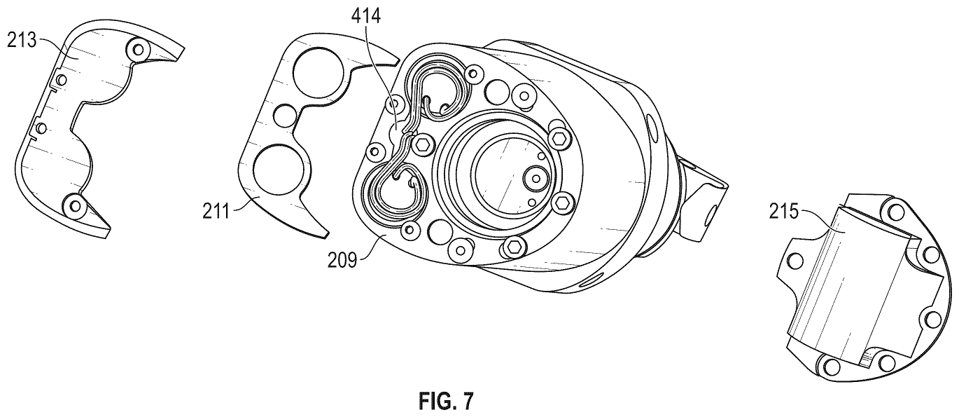

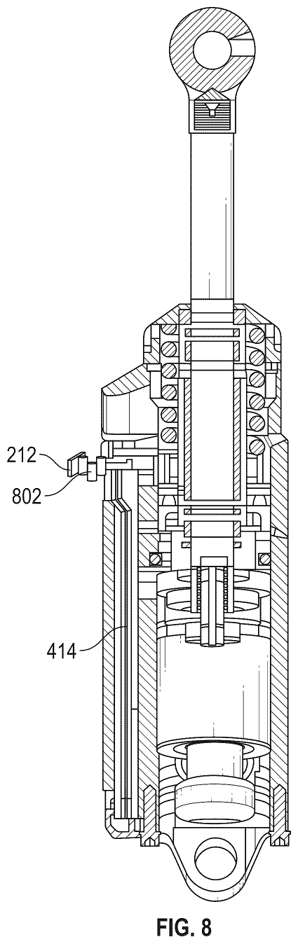

FIG. 6 is a bottom view of the cylinder body 205 (e.g., seamless single-piece, monolithic cylinder body). The hydraulic system 405 is housed within the piston bore 602. As described above with respect to FIG. 3, gearmotors 302 and 304 are housed within gearmotor sleeves 310 and 312, which in turn are housed within gearmotor bores 610 and 612, respectively. Electrical leads 314 extend within the wiring bore 414. FIG. 7 is an exploded view of the bottom of the cylinder body 205 showing the location of single sealing surface 209 and gasket 211 that can be placed over sealing surface 209 and sealed with cover 213, 215. There is additional seal at the sensor board 212 on the front side of the cylinder body. FIG. 7 also shows the single wiring bore 414, which is described in more detail with respect to FIG. 8. Embedding the motor-driven valves inside of the cylinder body 205 advantageously reduces the number of surfaces in contact with hydraulic fluid and reduces (e.g., minimizes) the number of seals required to inhibit (e.g., prevent) leaking and thus, reduces the likelihood of leaks occurring. Reducing the sealing surfaces also reduces (e.g., minimizes) the surfaces by which water can enter the system, making it easier to waterproof the unibody cylinder body 205 of the unibody cylinder construction 100.

FIG. 8 is a side cross-sectional view of the unibody cylinder construction 100. The single wiring bore 414 extends from the cylinder base 210 up the cylinder body 205 and is coupled to the sensor board 212 at a single connection 802. This construction promotes case of field replaceability. Because the hydraulic system 405 and valve-gear motor system 305 are contained in a single unit, the electrical connections required can be simplified to using the single connection 802. This single connection 802 allows for the cylinder to easily be replaced by the customer without requiring complex electrical connections and wiring.

Various modifications to the embodiments described in this disclosure will be readily apparent to those skilled in the art, and the generic principles defined herein may be applied to other embodiments without departing from the spirit or scope of this disclosure. Thus, the disclosure is not intended to be limited to the embodiments discussed herein but is to be accorded the widest scope consistent with the claims, the principles and the novel features disclosed herein. The word “example” is used exclusively herein to mean “serving as an example, instance, or illustration.” Any embodiment described herein as “example” is not necessarily to be construed as preferred or advantageous over other embodiments, unless otherwise stated.

Certain features that are described in this specification in the context of separate embodiments also may be embodied in combination in a single embodiment. Conversely, various features that are described in the context of a single embodiment also may be embodied in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the order shown or in sequential order, or that all illustrated operations be performed, to achieve desirable results. Additionally, other embodiments are within the scope of the following claims. In some cases, the actions recited in the claims may be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A prosthetic knee, comprising:
a unibody hydraulic cylinder extending between a proximal end and a distal end, comprising:
a central bore configured to movably receive a piston attached to a piston rod;
one or more bores configured to house a motorized valve system including a valve;
one or more pathways via which hydraulic fluid flows, and
one or more bores configured to house cabling connecting the motorized valve system to a circuit board,
wherein the central bore and one or more pathways are in fluid communication with each other via the valve.

2. The prosthetic knee of claim 1, wherein the one or more bores configured to house a motorized valve system are two bores, each configured to house a motorized valve system with a valve.

3. The prosthetic knee of claim 1, wherein the unibody hydraulic cylinder has a single sealing surface at one end thereof.

4. The prosthetic knee of claim 3, wherein the single sealing surface is at the distal end of the unibody hydraulic cylinder.

5. The prosthetic knee of claim 1, wherein the central bore, one or more bores configured to house the motorized valve system, one or more pathways and one or more bores configured to house cabling extend at least partially along parallel axes along a length of the unibody hydraulic cylinder.

6. The prosthetic knee of claim 1, wherein the central bore has a larger diameter than the one or more bores configured to house the motorized valve system.

7. The prosthetic knee of claim 1, wherein the one or more bores configured to house the motorized valve system has a larger diameter than the one or more pathways.

8. The prosthetic knee of claim 1, wherein the one or more pathways are two pathways.

9. A unibody hydraulic cylinder for a prosthetic knee, comprising:
a central bore configured to movably receive a piston attached to a piston rod;
one or more bores configured to house a motorized valve system including a valve;
one or more pathways via which hydraulic fluid flows, and
one or more bores configured to house cabling connecting the motorized valve system to a circuit board,
wherein the central bore and one or more pathways are in fluid communication with each other via the valve.

10. The unibody hydraulic cylinder of claim 9, wherein the one or more bores configured to house a motorized valve system are two bores, each configured to house a motorized valve system with a valve.

11. The unibody hydraulic cylinder of claim 9, further comprising a single sealing surface at one end of the unibody hydraulic cylinder.

12. The unibody hydraulic cylinder of claim 11, wherein the single sealing surface is at a distal end of the unibody hydraulic cylinder.

13. The unibody hydraulic cylinder of claim 9, wherein the central bore, one or more bores configured to house the motorized valve system, one or more pathways and one or more bores configured to house cabling extend at least partially along parallel axes.

14. The unibody hydraulic cylinder of claim 9, wherein the central bore has a larger diameter than the one or more bores configured to house the motorized valve system.

15. The unibody hydraulic cylinder of claim 9, wherein the one or more bores configured to house the motorized valve system has a larger diameter than the one or more pathways.

16. The unibody hydraulic cylinder of claim 9, wherein the one or more pathways are two pathways.

17. A device for actuation of a prosthetic, comprising:
an elongate hollow body defining one or more bores,
a hydraulic system, the hydraulic system comprising:
a piston;
hydraulic fluid;
an accumulator; and
one or more check valves, and
a motorized valve system, the motorized valve system comprising:
one or more motors, and
one or more valve cartridges, the one or more valve cartridges being operably connected to the one or more motors, wherein the hydraulic system and motorized valve system are housed within the one or more bores, at the elongate hollow body having a single sealing surface.

18. The device of claim 17, wherein the prosthetic is a prosthetic knee or a prosthetic foot.

19. The device of claim 17, wherein the accumulator is a spring-based accumulator.

20. The device of claim 17, wherein the one or more bores includes a first bore housing the hydraulic system, and at least one additional bore separate from the first bore and configured to house the motorized valve system.
